(19) United States
(12) Patent Application Publication
(10) Pub. No.: US 2009/0197960 A1
(43) Pub. Date: Aug. 6, 2009

(54) Method of Inhibiting Polyneuropathic Pain with 3-(2-Dimethylaminomethylcyclohexyl) Phenol

(75) Inventor: Thomas CHRISTOPH, Aachen (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 12/364,108

(22) Filed: Feb. 2, 2009

(30) Foreign Application Priority Data
Feb. 4, 2008 (EP) 08 002 013.4

(51) Int. Cl. A61K 31/195; A61K 31/135; A61P 29/00

(52) U.S. Cl. 514/567; 514/646

(57) ABSTRACT

A method of using 3-(2-dimethylaminomethylcyclohexyl)phenol or a pharmaceutically acceptable salt thereof as an active pharmaceutical ingredient for treating or inhibiting polyneuropathic pain, in particular in diabetic polyneuropathy.

### CROSS REFERENCE TO RELATED APPLICATIONS

This application claims priority from European patent application no. EP 08 002 013.4, filed Feb. 4, 2008, the entire disclosure of which is incorporated herein by reference.

### BACKGROUND OF THE INVENTION

The present invention relates to the treatment of polyneuropathic pain, in particular in diabetic polyneuropathy or chemically induced polyneuropathy, by the administration of 3-(2-dimethylaminomethylcyclohexyl)phenol, preferably of (1R,2R)-3-(2-dimethylaminomethylcyclohexyl)phenol or one of the pharmaceutically acceptable salts thereof.

Neuropathy is taken to mean a disease of the nervous system. In mononeuropathy, individual nerves are affected. Typical mononeuropathies are peripheral facial nerve paresis, carpal tunnel syndrome, carpi ulnaris lesion, radial nerve paresis and peroneal paresis. In contrast, in polyneuropathy many nerves are simultaneously affected. Around 3% of all people over 60 years of age suffer from a polyneuropathy.

Depending on the particular cause, motor, sensory or also vegetative nerves may be affected in polyneuropathy.

There are two main forms of polyneuropathy: peripheral polyneuropathy and autonomic polyneuropathy.

Peripheral polyneuropathy relates to that part of the nervous system which is under voluntary control, for example the nerves which are responsible for the sense of touch (sensory nerves) or muscle movement (motor nerves). Overall, impairment of the sensory nerve fibres which carry information from the periphery to the spinal cord and brain is to be observed earlier and in a more pronounced manner. The motor nerves, which are responsible for muscle movement, may, however, also be affected. Muscle spasms are frequent, but widespread paralysis somewhat rare.

Autonomic polyneuropathy involves the involuntary nervous system (sympathetic and parasympathetic branches) which are not under an individual's voluntary control. This vegetative nervous system for example controls the changing rate of the heartbeat or the movements of the gastrointestinal tract during digestion. Up to 50 percent of people who have been diabetic for 20 years suffer from an autonomic polyneuropathy. Any ill effects depend on which bodily organs are affected.

The disease may affect both the insulation of the nerves (myelin) and the nerve-cell process (axon). Both symmetrical and asymmetrical forms may occur. Symptoms are many and varied depending on what type of nerve fibre and part of the body are affected. Since the cell bodies (somatic part) of the sensory nerve cells are located in the ganglia close to the spinal cord and the nerve processes are supplied from there, it is the longest nerve fibres, which have to be supplied down to the toes, which most readily suffer damage. The disease process frequently begins with unpleasant paraesthesia in the toes. As the disease progresses, the patient frequently describes the distribution of the paraesthesia as “glove-like” or “sock-like”. Affected regions of the body may tingle and be unpleasantly numb, while more severe forms are manifested as polyneuropathic pain, which is frequently perceived as a burning pain. Erroneous sensations, such as for example a feeling of cold, heat or swelling, may also occur.

The signs and symptoms of polyneuropathy may be subdivided into three groups: (i) small-fibre sensory (for example burning pain, cutaneous hyperaesthesia, paraesthesia, shooting pain, loss of pain and temperature perception, loss of visceral pain perception, foot ulceration), (ii) large-fibre sensory (for example loss of vibration perception, ataxia due to loss of proprioception, loss of reflexes, reduced nerve conduction velocity) and (iii) autonomic (for example cardiac rhythm disturbances, resting tachycardia, loss of appropriate cardiac frequency adaptation, silent cardiac infarcts, cardiac insufficiency, orthostatic hypotension, gustatory sweating, hyperthermia, gastroparesis, neuropathic diarrhoea, constipation, bladder dysfunction, erectile dysfunction, retrograde ejaculation).

Depending on the type of nerve fibre affected, the following distinctions may be drawn:
fibre type Aα (I) (13-20 μm, myelinated): limb proprioception;
fibre type Aβ (II) (6-12 μm, myelinated): limb proprioception, vibration, pressure;
fibre type Aδ (III) (1-5 μm, myelinated): mechanical, sharp, stabbing pain, intense pressure on the skin (via mechanical nociceptors) and extreme temperature (via thermal nociceptors); - fibre type C (IV) (0.2-1.5 μm, unmyelinated): burning, long-lasting, diffuse pain, temperature-triggered pain, mechanical burning pain via polymodal nociceptors in the event of high intensity thermal or chemical stimuli.

Pain in polyneuropathy can only be combatted to a limited extent by administration of conventional analgesics (H. Chen et al., Mayo Clin. Proc. 2004, 79, 1533-45; M. Namaka et al., Clin. There. 2004, 26, 951-79). Placebo-controlled studies into the drug treatment of pain in polyneuropathy have revealed the following NNT values (Numbers Needed to Treat=the number of patients who must be treated with the particular medicine for a patient to experience more than 50% pain relief):

| | Active ingredient | NNT |
|---|---|---|
| Diabetic | Imipramine | 1.4 |
| polyneuropathy | Other tricyclic antidepressants | 2.4 |
| | Oextromethorphan | 1.9 |
| | Carbamazepine | 3.3 |
| | L-Dopa | 3.4 |
| | Tramadol | 3.4 |
| | Gabapentin | 3.7 |
| | Capsaicin | 5.9 |
| | Selective serotonin reuptake inhibitors | 6.7 |
| | Mexiletine | 10.0 |
| Post-herpetic | Tricyclic antidepressants | 2.3 |
| neuralgia | Oxycodone | 2.5 |
| | Gabapentin | 3.2 |
| | Capsaicin | 5.3 |

(cf. S. H. Sindrup et al., Pain, 1999, 83, 389-400).

The efficacy of a pharmaceutical substance in treating pain may in principle be manifested in various different respects. In addition to the alleviation of pain on a pain scale, an increase in the average pain threshold and/or a reduction in the number of tender points, which is particularly advantageous, may for example sometimes be observed.

### SUMMARY OF THE INVENTION

The object of the present invention is to provide medicaments for the treatment of polyneuropathic pain which have advantages over conventional medicaments.

A further object of the invention is to provide a method of treating or inhibiting polyneuropathic pain which will bring about the increased pain relief with the little or no side-effects.

These and other objects have been achieved by the invention as described and claimed hereinafter.

It has surprisingly been found that the active ingredient 3-(2-dimethylamino-methylcyclohexyl)phenol, in particular its (1R,2R)-stereoisomer or the pharmaceutically acceptable salts thereof exhibit excellent efficacy in the alleviation polyneuropathic pain. Experimental investigations have accordingly revealed that the efficacy of (1R,2R)-3-(2-dimethylaminomethylcyclohexyl)phenol in polyneuropathic pain in an animal model is approx. four times greater than in mononeuropathic pain. Since the active ingredient exhibits only slight side-effects, this opens up new possibilities for treatment which have considerable advantages over conventional treatment methods.

(1R,2R)-3-(2-Dimethylaminomethylcyclohexyl)phenol is also known by the INN “faxeladol” and has the following structure:

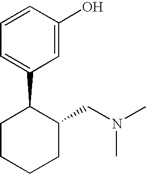

(1R,2R)-3-(2-Dimethylaminomethylcyclohexyl)phenol, some of the pharmaceutically acceptable salts thereof and use for treating specific types of pain are known from the prior art. Reference may for example be made in this connection to U.S. Pat. No. 5,733,936 and U.S. Pat. No. RE37,355 (=DE 195 25 137), US 2005/0182131 (=WO 2004/009067) US 2005/0277687 and US 2007/0249724 (=WO 2004/047823) and US 2007/0032551, US 2007/0032552 and US 2008/0255242 (=DE 10 2005 034 974).

The invention relates to the use of the active ingredient 3-(2-dimethylamino-methylcyclohexyl)phenol or one of the pharmaceutically acceptable salts thereof for producing a pharmaceutical composition for treating polyneuropathic pain.

A further aspect of the invention relates to 3-(2-dimethylaminomethylcyclo-hexyl)phenol or one of the pharmaceutically acceptable salts thereof for treating polyneuropathic pain. A still further aspect of the invention relates to a method for treating polyneuropathic pain comprising administering a pharmaceutically effective amount of 3-(2-dimethylaminomethylcyclohexyl)phenol or one of the pharmaceutically acceptable salts thereof to a subject in need thereof.

The active ingredient is preferably present in the form of the (1R,2R)-stereoisomer, i.e. as (1R,2R)-3-(2-dimethylaminomethyIcyclohexyl)phenol (faxeladol). However, in principle, the other stereoisomers, i.e. (1R,2S), (1S,2R) and (1S,2S) are also suitable.

The active ingredient may be present as a free base or as a pharmaceutically acceptable salt. Preferred pharmaceutically acceptable salts are salts of inorganic acids, for example the hydrochloride, hydrobromide, sulfate, hydrogensulfate, dihydrogenphosphate, hydrogenphosphate and phosphate; and salts of organic acids, for example the methanesulfonate, hexane-1-sulfonate, formate, acetate, oxalate, succinate, malate, tartrate, mandelate, fumarate, maleate, lactate, citrate, glutamate, saccharinate, sebacate, monomethyl sebacate, 5-oxo-prolinate, nicotinate, benzoate, aminobenzoate, methyl benzoate, trimethyl benzoate, □-lipoate, N-acetylglycinate, N-acetylalaninate, N-acetylcysteinate, N-acetylisoleucinate, N-acetylleucinate, N-acetylmethioninate, N-acetylphenylalaninate, N-acetylproline, N-acetylserine, N-acetylthreonine, N-acetyltyrosine, N-acetylvaline, acetylsalicylate, ascorbate, hippurate and aspartate. The active ingredient is particularly preferably present as a maleate salt.

The active ingredient is preferably formulated in a pharmaceutical composition. The pharmaceutical composition according to the invention may be, for example, solid, pasty or liquid. It preferably contains pharmaceutically acceptable auxiliary substances, for example fillers, binders, solvents, slip agents, and/or disintegrants. Which particular auxiliary substances are selected depends on how the pharmaceutical composition is to be administered. Suitable auxiliary substances are known to a person skilled in the art. Reference may be made in this connection, for example, to H. P. Fiedler, Lexikon der Hilfsstoffe für Pharmazie, Kosmetik und angrenzende Gebiete, Editio Cantor Aulendorf.

The active ingredient content of the pharmaceutical composition is preferably in the range from 0.001 to 99.999 wt. %, more preferably 0.1 to 95 wt. %, still more preferably 1.0 to 80 wt. %, most preferably 2.5 to 65 wt. % and in particular 5.0 to 50 wt. %, relative to the total weight of the pharmaceutical composition.

In addition to 3-(2-dimethylaminomethylcyclohexyl)phenol, the pharmaceutical composition according to the invention may optionally contain further active ingredients which may for example be selected from the group consisting of selective serotonin and norepinephrine reuptake inhibitors, α₂δ-ligands, tricyclic antidepressants, opioids and other analgesics. Preferably, however, a COX II inhibitor is not simultaneously present and preferably absolutely no further active ingredient is present.

A further aspect of the invention relates to a pharmaceutical composition as described above for treating polyneuropathic pain. A still further aspect of the invention relates to a method for treating polyneuropathic pain comprising administering the pharmaceutical composition as described above to a subject in need thereof.

In a preferred embodiment, the pharmaceutical composition is present as a dosage form. The dosage form according to the invention may, for example, be solid, pasty or liquid. The dosage form according to the invention is preferably formulated for systemic, parenteral, topical or local administration. The dosage form according to the invention is preferably formulated for oral or buccal administration. Other administration forms are, however, also possible, for example for buccal, sublingual, transmucosal, rectal, intralumbar, intraperitoneal, transdermal, intravenous, intramuscular, intragluteal, intracutaneous and subcutaneous administration.

Depending on the type of formulation, the dosage form preferably contains suitable additives and/or auxiliary substances. Suitable additives and/or auxiliary substances for the purposes of the invention are any substances known to a person skilled in the art for obtaining pharmaceutical formulations. The auxiliary substances selected and the quantities to be used are determined by how the dosage form is to be administered, i.e. orally, intravenously, intraperitoneally, intradermally, intramuscularly, intranasally, buccally or locally.

Preparations in the form of tablets, chewable tablets, sugar-coated tablets, capsules, granules, drops, juices and syrups are suitable for oral administration, while solutions, suspensions, easily reconstitutible dried preparations and sprays are suitable for parenteral, topical and inhalatory administration. Suppositories for rectal administration are a further possibility. Examples of suitable percutaneous administration forms include use in a depot in dissolved form, in a backing film or in a dressing, optionally with the addition of skin penetration promoters.

The dosage form may be present as a simple tablet and as a coated tablet (for example as a film-coated or sugar-coated tablet). The tablets are conventionally round and biconvex, but oblong shapes are also possible. Granules, spheroids, pellets or microcapsules which are packaged in sachets or capsules or press-moulded to form disintegrating tables are likewise possible.

Examples of auxiliary substances and additives for oral administration forms include disintegrants, slip agents, binders, fillers, mould release agents, optionally solvents, flavours, sugars, in particular carrier substances, diluents, dyes, antioxidants etc.

Waxes or fatty acid esters may be used for suppositories, while carriers, preservatives, suspension auxiliaries etc. may be used for parenteral administration forms.

Examples of useful auxiliary substances include water, ethanol, 2-propanol, glycerol, ethylene glycol, propylene glycol, polyethylene glycol, polypropylene glycol, glucose, fructose, lactose, sucrose, dextrose, molasses, starch, modified starch, gelatin, sorbitol, inositol, mannitol, microcrystalline cellulose, methylcellulose, carboxymethylcellulose, cellulose acetate, shellac, cetyl alcohol, polyvinylpyrrolidone, paraffins, waxes, natural and synthetic gums, gum arabic, alginates, dextran, saturated and unsaturated fatty acids, stearic acid, magnesium stearate, zinc stearate, glyceryl stearate, sodium lauryl sulfate, edible oils, sesame oil, coconut oil, peanut oil, soya oil, lecithin, sodium lactate, polyoxyethylene and polyoxypropylene fatty acid esters, sorbitan fatty acid esters, sorbic acid, benzoic acid, citric acid, ascorbic acid, tannic acid, sodium chloride, potassium chloride, magnesium chloride, calcium chloride, magnesium oxide, zinc oxide, silicon dioxide, titanium oxide, titanium dioxide, magnesium sulfate, zinc sulfate, calcium sulfate, potash, calcium phosphate, dicalcium phosphate, potassium bromide, potassium iodide, talcum, kaolin, pectin, crospovidone, agar and bentonite.

These medicaments and pharmaceutical compositions according to the invention are produced using means, devices, methods and processes known from the prior art of pharmaceutical technology, as are described, for example, in “Remington's Pharmaceutical Sciences”, ed. A. R. Gennaro, 17th edition, Mack Publishing Company, Easton, Pa. (1985), in particular in part 8, sections 76 to 93.

For example, for a solid formulation, such as a tablet, the active ingredient of the medicament may be granulated with a pharmaceutical carrier, for example conventional tablet ingredients, such as maize starch, lactose, sucrose, sorbitol, talcum, magnesium stearate, dicalcium phosphate, or pharmaceutically acceptable gums, and pharmaceutical diluents, such as for example water in order to form a solid composition which contains the active ingredient in homogeneously dispersed form. Homogeneously dispersed is here taken to mean that the active ingredient is uniformly dispersed throughout the entire composition, such that the latter may straightforwardly be subdivided into equally active unit dosage forms, such as tablets, capsules or sugar-coated tablets. The solid composition is then subdivided into unit dosage forms. The tablets or pills may also be coated or compounded in another manner in order to provide a dosage form with delayed release. Suitable coating compositions are, inter alia, polymeric acids and mixtures of polymeric acids with materials such as, for example shellac, cetyl alcohol and/or cellulose acetate.

In preferred embodiments of the invention, the dosage form according to the invention is formulated for administration once, twice or three times daily.

Delayed release of the active ingredient may, for example, be achieved by controlled-release action with the assistance of a matrix, a coating or osmotic release systems (cf. for example US 2006/0121113 (=WO 2005/009329) the disclosure of which is incorporated herein by reference).

Under in vitro conditions, the dosage form used according to the invention preferably releases after 1 h at least 5 wt. %, more preferably at least 10 wt. %, still more preferably at least 15 wt. %, most preferably at least 20 wt. % and in particular at least 25 wt. % of the active ingredient originally present in the dosage form. Under in vitro conditions, the dosage form according to the invention preferably releases after 1 h at most 95 wt. %, more preferably at most 90 wt. %, still more preferably at most 85 wt. %, most preferably at most 80 wt. % and in particular at most 75 wt. % of the active ingredient originally present in the dosage form. Suitable methods for determining the in vitro release rate are known to a person skilled in the art. Determination preferably proceeds under sink conditions at 75 rpm in a buffer (according to Ph. Eur.) at a pH value of 6.8 at 37° C. and with UV spectroscopic detection with the assistance of a paddle stirrer apparatus or the rotating basket method.

The dosage form according to the invention contains the active ingredient preferably in a dosage in the range from 1.0 to 1000 mg, more preferably 5.0 to 900 mg, still more preferably 10 to 800 mg, most preferably 15 to 700 mg and in particular 20 to 600 mg, in each case relative to the free base. Usually, 0.1 to 5000 mg/kg, in particular 1 to 500 mg/kg, preferably 2 to 250 mg/kg of body weight are administered. It is, however, likewise preferred and usual also to administer from 0.01 to 5 mg/kg, preferably 0.03 to 2 mg/kg, in particular 0.05 to 1 mg/kg.

In a preferred embodiment:
the dosage form is formulated for oral administration; and/or
the dosage form is a solid and/or press-moulded and/or film-coated dosage form; and/or
the dosage form releases the active ingredient from a matrix in delayed manner; and/or
the dosage form contains the active ingredient in a quantity of 0.001 to 99.999 wt. %, more preferably 0.1 to 99.9 wt. %, still more preferably 1.0 to 99.0 wt. %, still more preferably 2.5 to 80 wt. %, most preferably 5.0 to 50 wt. % and in particular 7.5 to 40 wt. %, relative to the total weight of the dosage form; and/or
the dosage form contains a pharmaceutically acceptable carrier and/or pharmaceutically acceptable auxiliary substances; and/or
the dosage form has a total mass in the range from 25 to 2,000 mg, more preferably 50 to 1,800 mg, still more preferably 60 to 1,600 mg, still more preferably 70 to 1,400 mg, most preferably 80 to 1,200 mg and in particular 100 to 1,000 mg, and/or
the dosage form is selected from the group consisting of tablets, capsules, pellets and granules.

A further aspect of the invention relates to a dosage form as described above for treating polyneuropathic pain. A still further aspect of the invention relates to a method for treating polyneuropathic pain comprising administering the dosage form as described above to a subject in need thereof.

The active ingredient 3-(2-dimethylaminomethylcyclohexyl)phenol and the pharmaceutically acceptable salts thereof is suitable for treating polyneuropathic pain. The pain is preferably peripheral polyneuropathic pain or central polyneuropathic pain. The polyneuropathy or the polyneuropathic pain is preferably acute (up to four weeks), sub-acute (four to eight weeks) or chronic (more than eight weeks). The motor, sensory, autonomic, sensomotor or central nervous system is preferably affected in the polyneuropathy. The symptoms are preferably distributed symmetrically or asymmetrically. The pain may be slight, moderate, moderately severe, severe or very severe. The neuropathic pain scale (NPS) may be used as a measure (cf. B. S. Galer et al., Neurology 1997, 48, 332-8).

Examples of causes of peripheral polyneuropathic pain include diabetic polyneuropathy, post-herpetic neuralgia, radiculopathy, post-traumatic neuralgia, polyneuropathy induced by chemical substances, for example by chemotherapy, phantom pain in the limbs, complex regional syndrome, HIV-induced sensory polyneuropathy and alcoholic polyneuropathy. Examples of causes of central polyneuropathic pain are compressive myelopathy due to narrowed canal stenosis, post-traumatic spinal pain, stroke pain, post-ischaemic myelopathy, radiation-induced myelopathy, multiple sclerosis-induced myelopathy and HIV-induced myelopathy.

In one preferred embodiment, the polyneuropathy causing the polyneuropathic pain is associated with a complaint selected from the group consisting of diabetes, diabetes mellitus, vasculitis, uraemia, hypothyroidism, alcohol abuse, post-herpetic neuralgia, idiopathic neuropathy, chronic inflammatory demyelinating neuropathy, multifocal motor neuropathy, hereditary polyneuropathy, Guillain-Barré syndrome, poisoning [for example by alcohol, heavy metals {in particular Pb, Hg, As}, hydrocarbons, following chemotherapy with cytostatic agents], porphyria, infectious diseases, neoplastic diseases [for example myeloma, amyloidosis, leukaemia, lymphomas], pernicious anaemia, vitamin E deficiency, Refsum's disease, Bassen-Kornzweig syndrome, Fabry's disease, vasculitis and amyloidosis. Diabetic polyneuropathy and post-herpetic neuralgia are particularly preferred. If an infectious disease is involved, this is preferably selected from the group consisting of mononucleosis, ehrlichiosis, typhus, diphtheria, leprosy, HIV, lues and borreliosis.

The polyneuropathic pain is preferably pain which is caused by a polyneuropathy as defined in ICD-10 (International Statistical Classification of Diseases and Related Health Problems, issued by WHO, preferably 2008 edition). The polyneuropathy is preferably selected from paraneoplastic polyneuropathy, hereditary and idiopathic neuropathy [G60], polyneuritis [G61], other polyneuropathies [G62], polyneuropathy in diseases classified elsewhere [G63], neuralgia NOS [M79.2-], neuritis NOS [M79.2-], pregnancy-related peripheral neuritis [026.83] and radiculitis NOS [M54.1-].

If it is a hereditary or idiopathic neuropathy [G60], this is preferably selected from the group consisting of hereditary sensomotor neuropathy [G60.0] (Charcot-Marie-Tooth-Hoffmann syndrome, Dejerine-Sottas disease, hereditary sensomotor neuropathy, types l-IV, hypertrophic neuropathy of infancy, peroneal muscular atrophy (axonal type) (hypertrophic form), Roussy-Levy syndrome); Refsum's disease [G60.1]; neuropathy in association with hereditary ataxia [G60.2]; idiopathic progressive neuropathy [G60.3]; other hereditary and idiopathic neuropathies [G60.8] (Morvan's disease, Nelaton's syndrome, sensory neuropathy: dominantly inherited or recessively inherited); and hereditary and idiopathic neuropathy, unspecified [G60.9].

If it is a polyneuritis [G61], this is preferably selected from the group consisting of Guillain-Barré syndrome (polyradiculoneuropathy) [G61.0] (acute (post-)infective polyneuritis); serum polyneuropathy [G61.1], other inflammatory polyneuropathies [G61.8] and inflammatory polyneuropathy, unspecified [G61.9].

If it is an “other” polyneuropathy [G62], this is preferably selected from the group consisting of drug-induced polyneuropathy [G62.0], alcoholic polyneuropathy [G62.1], polyneuropathy due to other toxic agents [G62.2], other specified polyneuropathies [G62.8] (radiation-induced polyneuropathy, critical illness polyneuropathy [G62.80], other specified polyneuropathies [G62.88]) and polyneuropathy, unspecified [G62.9] (neuropathy NOS).

If it is a polyneuropathy in diseases classified elsewhere [G63], this is preferably selected from the group consisting of polyneuropathy in infectious and parasitic diseases classified elsewhere [G63.0] (polyneuropathy in diphtheria [A36.8†], infectious mononucleosis [B27.-†], leprosy [A30.-†], Lyme disease [A69.2†], mumps [B26.8†], post-herpetic [B02.2†], late syphilis [A52.1†], congenital late syphilis [A50.4†], tuberculosis [A17.8†]); polyneuropathy in neoplastic disease [G63.1] [C00-D48†]; diabetic polyneuropathy [G63.2] [E10-E14†, fourth character 0.4]; polyneuropathy in other endocrine and metabolic diseases [G63.3] [E00-E07†, E15-E16†, E20-E34†, E70-E89†]; polyneuropathy in nutritional deficiency [G63.4] [E40-E64†]; polyneuropathy in systemic connective tissue disorders [G63.5] [M30-M35†]; polyneuropathy in other musculoskeletal disorders [G63.6] [M00-M25†, M40-M96†]; and polyneuropathy in other diseases classified elsewhere [G63.8] (uraemic neuropathy [N18.8†]).

In one particularly preferred embodiment, the polyneuropathic pain is associated with diabetic polyneuropathy [G63.2]. More than the half of all diabetics develop nerve damage (polyneuropathy) once they have suffered from diabetes for longer than ten years. Taking all type 1 and type 2 diabetics together, some 30 percent suffer in this manner.

In another particularly preferred embodiment, the polyneuropathic pain is caused by toxic agents induced (chemically induced polyneuropathy), preferably by drugs (for example chemotherapeutic agents) [G62.0] or alcohol [G62.1]. Various toxic agents are known which can induce polyneuropathy. Examples of drugs are cisplatin, didanosine, stavudine and zalcitabine.

### EXAMPLES

The following examples illustrate the invention in further detail but are not intended to limit the scope of the invention.

Male Sprague Dawley rats (140-180 g, Janvier, France) were kept under standard conditions (06.00-18.00 light, 18.00-06.00 darkness; 20-24° C. room temperature, 35-70% relative humidity; tap water and standard feed (ad libitum)) in groups of five animals in Macrolon type 4 cages.

### Comparative Example—Mononeurolathic Pain

The investigation was carried out according to Bennett et al., Pain, 1988, 33, 87-107.

Under pentobarbital anaesthesia (Narcocen, 60 mg/kg i.p., Merial GmbH, Germany), four loose ligatures were placed unilaterally on the right ischial nerve. On the paw innervated by the damaged nerve, the animals developed hypersensitivity which, after one week's convalescence, was quantified over a period of approx. four weeks using a cold metal plate at 4° C. (cold allodynia). The animals were observed on this plate for a period of 2 min. and the number of withdrawal responses by the damaged paw was measured. The action of the substance, relative to the preliminary value before administration of the substance, was determined over a period of one hour at four different times (15, 30, 45, 60 min after administration). The inhibition of cold allodynia at the individual measurement times was stated in percent relative to the individual pretest (% MPE), with the pretest corresponding to 0% MPE and complete inhibition (0 withdrawal responses per 2 min) to 100% MPE. Group size was n=10. The significance of a substance's action was determined on the basis of the percentage inhibition values relative to the vehicle group by bifactorial analysis of variance and post hoc Bonferroni analysis, the ED₅₀ value by linear regression analysis for the individual measurement points or the area under the curve (AUC).

(1R,2R)-3-(2-Dimethylaminomethylcyclohexyl)phenol (10-31.6 mg/kg i.p.) resulted in dose-dependent inhibition of cold allodynia. The minimally active dose at which a statistically significant inhibition was obtained was 21.5 mg/kg i.p. The maximum effect was 80% MPE 15 min after administration of 31.6 mg/kg, the ED₅₀ value (95% VB) was 17.6 (14.1-21.4) mg/kg 30 min after administration.

The measured results are summarized in the following table (inhibition of cold allodynia in % MPE;*<0.05 vs. vehicle; N.S. not significant vs. vehicle):

| Dose | (% MPE) | 15 min | 30 min | 45 min | 60 min |
|---|---|---|---|---|---|
| 10 | Mean | 22.16 | 20.93 | 8.60 | 9.23 |
| | SEM | 9.14 | 10.08 | 7.05 | 7.74 |
| | Significance | N.S. | N.S. | N.S. | N.S. |
| 21.5 | Mean | 56.62 | 61.06 | 34.96 | 16.33 |
| | SEM | 8.65 | 7.25 | 10.01 | 11.37 |
| | Significance | * | * | N.S. | N.S. |
| 31.6 | Mean | 80.03 | 79.15 | 61.68 | 21.18 |
| | SEM | 10.96 | 7.25 | 10.16 | 9.10 |
| | Significance | * | * | * | N.S. |

### Example—Polyneuropathic Pain (Pre-Clinical Trial)

The investigation was carried out according to Authier et al., Neuroreport, 1999, 10, 965-8.

Rats were treated on five days (day 4, 6, 8, 10, 12) with vehicle (0.9% NaCl) or vincristine (200 pg/kg i.v.) (1 ml/kg), which resulted in a cumulative vincristine dose of 1 mg/kg. Three days after the final vincristine treatment, the animals had developed hypersensitivity to cold (cold allodynia) which continued over a period of three weeks. For testing, the animals were placed under a plastic cover on a grating and, after habituation, cold allodynia was quantified. To this end, a drop of acetone (10 μl) was carefully applied onto a hind paw with a syringe and a thin plastic tube. The number of induced withdrawal responses (shaking, stamping or licking) was recorded over a period of 30 sec. The cumulative number from 5 stimulations (at 5 min intervals from one another) was determined before and at different times after administration of the substance or the vehicle. The percentage inhibition value (% MPE) was determined in comparison with the particular pretest (0% MPE) and with the weekly vehicle control group (100% MPE=0 withdrawal responses). The size of the group was usually n=10. The significance of a substance's action was determined on the basis of the percentage inhibition value relative to the vehicle group by means of bifactorial analysis of variance and post hoc Bonferroni analysis, the ED₅₀ value by linear regression analysis for the individual measurement points.

(1R,2R)-3-(2-Dimethylaminomethylcyclohexyl)phenol (fexeladol) (10-21.5 mg/kg i.p.) resulted in dose-dependent inhibition of cold allodynia. The minimally active dose at which a statistically significant inhibition was obtained was 4.64 mg/kg i.p. The maximum effect was 74% MPE 30 min after administration of 21.5 mg/kg, the ED₅₀ value (95% VB) was 5.7 (3.3-11.1). The measured results are summarised in the following table (inhibition of cold allodynia in % MPE;*<0.05 vs. vehicle; N.S. not significant vs. vehicle):

| Dose | (% MPE) | 30 min | 180 min |
|---|---|---|---|
| 10 | Mean | −28.9 | −22.2 |
| | SEM | 23.8 | 23.0 |
| | Significance | N.S. | N.S. |
| 2.15 | Mean | 48.4 | −37.7 |
| | SEM | 8.0 | 19.3 |
| | Significance | N.S. | N.S. |
| 4.64 | Mean | 67.9 | 22.6 |
| | SEM | 5.3 | 12.2 |
| | Significance | * | N.S. |
| 10 | Mean | 58.7 | 20.8 |
| | SEM | 17.7 | 13.8 |
| | Significance | N.S. | N.S. |
| 21.5 | Mean | 74.2 | 15.2 |
| | SEM | 12.3 | 10.0 |
| | Significance | * | N.S. |

The foregoing experimental results demonstrate that (1R,2R)-3-(2-dimethyl-aminomethylcyclohexyl)phenol (faxeladol) brings about dose-dependent inhibition of cold allodynia in mono- and polyneuropathic pain. A significant difference with regard to effectiveness may be observed in the two animal models. Whereas in the polyneuropathic pain model significant inhibition is already observed at a dose of 4.64 mg/kg i.p., in the mononeuropathic pain model significant inhibition does not occur until a dose of 21.5 mg/kg i.p., i.e. at a dosage which is more than four times higher. Similar behavior is observed with the ED₅₀ value which, at the same time after administration (30 min), amounts to a dose of 5.7 mg/kg i.p. in the polyneuropathic pain model and a dose of 17.6 mg/kg i.p. in the mononeuropathic pain model, activity thus being greater by approx. a factor of 3 in the polyneuropathic pain model.

These preclinical data demonstrate that (1R,2R)-3-(2-dimethylaminomethyl-cyclohexyl)phenol (faxeladol) or the pharmaceutically acceptable salts thereof are particularly suitable for treating polyneuropathic pain.

### Example—Polyneuropathic Pain (Clinical Trial)

A crossover multiple-dose trial was performed in order to assess the analgesic efficacy and safety of oral (1R,2R)-3-(2-dimethylaminomethylcyclohexyl)-phenol (faxeladol) prolonged release (PR) compared with placebo in subjects with painful polyneuropathy of mixed origin. The trial was designed as a randomized, multicenter, double-blind, placebo-controlled, 3-way crossover, dose-titration, Phase II trial. Sixty-four (64) subjects with painful polyneuropathy of mixed origin (diabetic, idiopathic, alcoholic or drug-induced neuropathy) with symptoms present for more than 6 months were randomized and treated.

The following investigational medicinal products were administered:

| | faxeladol PR | Placebo |
|---|---|---|
| Dose | 60 mg | |
| Mode of | Oral | Oral |
| administration | | |
| Duration of treatment | Up to 4 weeks | Up to 4 weeks |

Efficacy of faxeladol PR 120 mg to 240 mg was demonstrated in subjects with painful polyneuropathy of mixed origin. Faxeladol PR was superior to placebo regarding the reduction of the average pain intensity. The analgesic efficacy of faxeladol PR in subjects with neuropathic pain of mixed origin was clearly demonstrated.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A method of treating or inhibiting polyneuropathic pain in a subject, said method comprising administering to said subject a pharmacologically effective amount of 3-(2-dimethylaminomethylcyclohexyl)phenol or a pharmaceutically acceptable salt thereof.

2. A method as claimed in claim 1, wherein said 3-(2-dimethylaminomethylcyclo-hexyl)phenol or a pharmaceutically acceptable salt thereof is present as (1R,2R)-3-(2-dimethylaminomethylcyclohexyl)phenol or a salt thereof.

3. A method as claimed in claim 1, wherein the 3-(2-dimethylaminomethylcyclo-hexyl)phenol or a pharmaceutically acceptable salt thereof is present as 3-(2-dimethylaminomethylcyclohexyl)phenol maleate.

4. A method as claimed in claim 1, wherein the 3-(2-dimethylaminomethylcyclo-hexyl)phenol or salt thereof is administered as a solid or liquid dosage form.

5. A method as claimed in claim 4, wherein the 3-(2-dimethylaminomethylcyclo-hexyl)phenol or salt thereof is administered orally as a solid or liquid dosage form.

6. A method as claimed in claim 1, wherein the 3-(2-dimethylaminomethylcyclo-hexyl)phenol or salt thereof is administered once per day.

7. A method as claimed in claim 1, wherein the 3-(2-dimethylaminomethylcyclo-hexyl)phenol or salt thereof is administered twice per day.

8. A method as claimed in claim 1, wherein the 3-(2-dimethylaminomethylcyclo-hexyl)phenol or salt thereof is administered three times per day.

9. A method as claimed in claim 1, wherein the 3-(2-dimethylaminomethylcyclo-hexyl)phenol or salt thereof is administered as a dosage form which, under in vitro conditions, releases at least 20 wt. % of the 3-(2-dimethylaminomethylcyclohexyl)-phenol or salt thereof originally contained in the dosage form after 1 hour.

10. A method as claimed in claim 4, wherein said dosage form contains the 3-(2-dimethylaminomethylcyclohexyl)phenol or salt thereof in a dosage of from 15 to 700 mg, relative to the free base.

11. A method as claimed in claim 1, wherein said polyneuropathic pain is peripheral polyneuropathic pain or central polyneuropathic pain.

12. A method as claimed in claim 1, wherein said polyneuropathic pain is associated with diabetic polyneuropathy or post-herpetic neuralgia.
